(19) United States
(12) Patent Application Publication
(10) Pub. No.: US 2002/0160482 A1
(43) Pub. Date: Oct. 31, 2002

(54) Methods for protein purification

(76) Inventors: Lars Abrahmsen, Bromma (SE); Joakim Nilsson, Danderyd (SE)

Correspondence Address: JANIS K. FRASER, PH.D., FISH & RICHARDSON P.C., 225 Franklin Street, Boston, MA, 02110-2804, US

(21) Appl. No.: 10/081,408

(22) Filed: Feb. 21, 2002

Related U.S. Application Data
(60) Provisional application No. 60/272,247, filed on Feb. 28, 2001.

(30) Foreign Application Priority Data
Feb. 23, 2001 (SE) 0100625-3

(51) Int. Cl. C12N 9/06; C07H 21/04; C12P 21/02; C12N 5/06

(52) U.S. Cl. 435/191; 435/69.1; 435/325; 435/320.1; 536/23.2

(57) ABSTRACT

The present invention relates to a recombinant construct comprising a nucleotide sequence encoding a fusion protein comprising a soluble form of human SSAO (Semicarbazide-Sensitive Amine Oxidase), a secretable fusion partner, a signal peptide; and a protease cleavage site. The said construct is useful in methods for purification of a soluble form of human SSAO.

### CROSS REFERENCE TO RELATED APPLICATIONS

This application claims priority from Swedish Patent Application No, 0100625-3, filed Feb. 23, 2001, and U.S. Provisional Patent Application Serial No. 60/272,247, filed Feb. 28, 2001. These applications are incorporated herein by reference in their entirety.

### TECHNICAL FIELD

The present invention relates to a recombinant construct comprising a nucleotide sequence encoding a fusion protein comprising a soluble form of human semicarbazide-sensitive amine oxidase (SSAO), a secretable fusion partner, a signal peptide, and a protease cleavage site. The invention also relates to methods for purification of a soluble form of human SSAO, said methods utilizing the recombinant construct.

### BACKGROUND ART

Semicarbazide-sensitive amine oxidase (SSAOs) belong to the copper-containing amine oxidase family of enzymes (CuAO; EC. 1.4.3.6) and are widely distributed among both eukaryotic and prokaryotic organisms (Buffoni, 1993). The physiological role of this abundant enzyme is essentially unknown and endogenous substrates with high affinity have so far not been identified, although benzylamine is an artificial high-affinity substrate (Buffoni, 1993; Callingham et al., 1995; Lyles, 1996, Hartmann and McIntire, 1997; Holt et al., 1998). In humans high SSAO activity is found in vascular smooth muscle cells (Lewinsohn 1984; Nakos and Gossrau, 1994; Yu et al., 1994; Lyles and Pino, 1998; Jaakkola et al., 1999). SSAO activity has also been detected in smooth muscle cells of non-vascular type and in endothelial cells (Lewinsohn, 1984; Castillo et al., 1998; Jaakkola et al., 1999). Small amounts of SSAO protein is also found in blood showing similar properties compared to the tissue-bound form (Yu and Zuo, 1993; Yu et al., 1994; Kurkijärvi et al., 1998).

Many studies have demonstrated that SSAO activity in blood plasma is elevated in several human conditions such as heart failure, atherosclerosis and diabetes (Lewinsohn, 1984; Boomsma et al., 1997; Ekblom, 1 p98; Boomsma et al., 1999; Meszaros et al., 1999). The mechanism(s) underlying these alterations of enzyme activity are currently uncharacterized. It has been suggested that reactive aldehydes and hydrogen peroxide produced by endogenous amine oxidases could be causative or contribute to the progression of cardiovascular diseases, and that inhibition of SSAO activity in diabetics might decrease vascular complications (Ekblom, 1998).

Recently it was found that the cDNA sequence of human SSAO (Zhang and McIntire, 1996) is identical to the vascular adhesion protein 1 (VAP-1), which participates in lymphocyte recirculation by mediating the binding of lymphocytes to peripheral lymph node vascular endothelial cells (Smith et al., 1998; see also WO 98/53049). The cDNA sequence of SSAO/VAP-1 is deposited under GenBank Accession Nos. U39447 and NM_{—}003734 (SEQ ID NO: 1). VAP-1 has also been found to be up-regulated on the endothelial cell surface under inflammatory conditions (Smith et al., 1998). However, the adhesive properties of SSAO have only been found in endothelial cells. In smooth muscle cells, SSAO does not support binding of lymphocytes (Jaakola et al., 1999). DNA-sequence analysis, structural modeling and experimental data suggest that human SSAO is a homodimeric glycoprotein consisting of two 90-100 kDa subunits anchored to the plasma membrane by a single N-terminal membrane spanning domain (Morris et al., 1997; Smith et al., 1998; Salminen et al., 1998).

No reports have so far been published regarding the purification of a recombinant mammalian SSAO or purification to near homogeneity of larger amounts of a human SSAO from a natural source. One report has described the use of a FLAG peptide fused to the N-terminal end of full-length human SSAO for detection purposes, but no results were presented regarding its use for purification of the human SSAO protein (Smith et al., 1998). Monoclonal antibodies have been used to immunoaffinity purify small amounts of human SSAO from serum and tissue homogenates for immunoblotting (Smith et al., 1998; Kurkijärvi et al., 1998). Consequently, there is a need for alternative methods for the purification of human SSAO in significant amounts.

Glutathione S-transferase (GST) from Schistosoma japonicum is a homodimeric cytoplasmic enzyme that can be purified by affinity chromatography using immobilized cofactor glutathione, followed by competitive elution using reduced glutathione (GSH). Taking advantage of this specific interaction, a gene fusion system for E. coli intracellular expression was developed by Smith and co-workers (Smith & Johnson, 1988; see also WO 88/09372) to facilitate detection and purification of recombinant proteins fused to GST. A potential drawback with using GST as fusion partner is the possibility that the free cysteines on its surface can crosslink with free cysteines on e.g. the fused target protein when exposed to an oxidizing environment. To minimize this risk and to allow for secretion of GST-fusion proteins a mutant form of GST was recently developed, which retain both its ability to form homo-dimers and its enzyme activity (Tudyka and Skerra, 1997). The homo-dimerization propensity of GST can be used to provoke dimerization of the fused target protein e.g. for the purpose of increased avidity effects (Tudyka and Skerra, 1997).

Alternative homodimeric fusion partners described in the literature are e.g. the Fc region of immunoglobulins (Hollenbaugh et al., 1992; Sakurai et al., 1998; Lo et al., 1998; Dwyer et al., 1999) and leucine zippers such as GCN4 (Rieker and Hu, 2000; Müller et al., 2000). Several different proteins have been fused to these homodimeric protein domains for different purposes e.g. to increase avidity (Dwyer et al., 1999; Muller et al., 2000) and to restore high-affinity DNA binding of truncated DNA-binding proteins (Rieker and Hu, 2000). Fc-fusion protein can be purified by protein A-affinity chromatography involving elution with low pH buffers (Sakurai et al., 1998; Lo et al., 1998), which may decrease activity of the fused target protein (Gräslund et al., 1997). Another problem associated with using Fc as fusion partner is the use of serum for cell growth, which complicate detection and purification of secreted Fc-fusions since serum contains large amounts of immunoglobulins (Sakurai et al., 1998). The leucine zipper GCN4 has mostly been used as fusion partner for proteins expressed in E. coli (Miller et al., 2000) and an affinity-tag might has to be fused to facilitate purification.

### BRIEF DESCRIPTION OF THE DRAWINGS

FIG. 1 is a schematic illustration of a GST-SSAO DNA construct (SEQ ID NO: 19) encoding a fusion protein (SEQ ID NO: 20). The three cysteine to serine mutations (residues 85, 138, and 178 according to the sequence having GenBank™ Accession No. M 14654) in the GST fusion partner are shown with boldface letters. Boxed sequence represents the recognition sequence for the 3C-protease.

FIG. 2 is an overview of an SSAO purification process. The determined specific activities for each purification step are indicated.

FIG. 3 is a schematic illustration of the GST-SSAO expression vector designated pMB887.

### DISCLOSURE OF THE INVENTION

According to the present invention, it has unexpectedly been found that soluble human SSAO can be produced in milligram quantities in a purification system utilizing a fusion partner capable of enabling dimerization of soluble SSAO. Consequently, in a first aspect this invention provides a recombinant construct comprising a nucleotide sequence encoding a fusion protein comprising:

(i) a soluble form of human SSAO;

(ii) a secretable fusion partner enabling dimerization of SSAO;

(iii) a signal peptide allowing for secretion of a polypeptide from a host cell into the culture medium; and

(iv) a protease cleavage site located between the human SSAO variant and the fusion partner.

As will be understood by the skilled person, the recombinant construct can optionally comprise one or more nucleotide sequences coding for spacer amino acid sequences of various lengths. Such spacer sequences could be used in order to increase the flexibility within the fusion protein, or to increase the space between protein domains so that folding can take place independently of adjacent domains. Further, spacers could be useful for increasing the accessibility for a protease to cleave at an introduced cleavage recognition sequence.

The soluble form of human SSAO is preferably lacking the membrane spanning portion of wild-type human SSAO. The membrane spanning portion of the SSAO polypeptide is known in the art (Morris et al., 1997; Holt et al., 1998; Smith et al., 1998) and is essentially set forth as amino acids 5 to 27, in particular amino acids 6 to 26, of SEQ ID NO: 2.

The amino acid sequence for human SSAO, excluding the membrane spanning portion, preferably comprises, or essentially consists of, positions 29 to 763 in SEQ ID NO: 2. However, the skilled person will understand that a part of the membrane-spanning portion could be included in the SSAO polypeptide while the polypeptide would still retain its essentially soluble properties. Consequently, the amino acid sequence for human SSAO could comprise e.g. positions 27 to 763, or 28 to 763, of SEQ ID NO: 2, including fragments thereof having substantially the biological activities of human SSAO. Further, the term “human SSAO polypeptide” is intended to encompass mutants and naturally occurring variants of human SSAO, either having retained enzymatic activity or protein interaction (e.g. adhesion function), or designed to facilitate structural studies (e.g. improved properties for crystallization), or mutated to facilitate studies of structure/function relationships (which also includes inactive mutants).

The fusion partner can be fused to the C-terminal or N-terminal portion of the human SSAO polypeptide. It is envisaged that the fusion protein could comprise more than one fusion partner, for instance one fused to the N-terminal and one fused to the C-terminal part of SSAO. An additional fusion partner could be an additional affinity tag, or a reporter protein such as Enhanced Green Fluorescent Protein (EGFP).

A large number of different gene fusion systems and fusion partners have been described. In such systems, different types of interactions, such as enzyme-substrate, bacterial receptor-serum protein, polyhistidines-metal ion, and antibody-antigen, have been utilized (Uhlén et al., 1992). Various gene fusion systems for affinity purification are also known in the art. Examples of fusion partners used in such systems (for reviews, see e.g. Nilsson et al., 1997; or Sheibani, 1999) comprise staphylococcal Protein A and its derivative Z; the albumin-binding protein from streptococcal Protein G; glutathione S-transferase (GST); polyhistidine tags; biotinylated affinity tags (e.g Biotin AviTag); E. coli maltose-binding protein; cellulose binding domains; the FLAG peptide; and Strep-tag. Alternative systems may be engineered using protein scaffolds for generation of novel ligand receptors (see Skerra, 2000, and references therein). These novel binding proteins, e.g. affibodies, may then be useful as fusion partners for different applications (Nygren and Uhlén, 1997; Nord et al., 1997).

According to this invention, the said fusion partner should enable dimerization of SSAO. A suitable fusion partner is glutathione S-transferase (GST), because of its propensity to dimerize and because the purification procedure has the potential to be performed under mild conditions using chromatography media with immobilized glutathione (e.g. from Amersham Pharmacia Biotech, Uppsala, Sweden). In addition, GST can conveniently be detected either by its enzymatic activity or by the use of GST specific antibodies or glutathione, using commercially available GST detection systems (e.g. from Amersham Pharmacia Biotech). The fusion partner could also be a functionally equivalent variant of GST, having retained propensity for dimerization and having binding properties allowing affinity purification. The said fusion partner is more preferably a variant of S. japonicum GST (GenBank Accession No. M14654; SEQ ID NOS: 3 and 4), designed for secretion out of the host cell, having one or more of the cysteine residues in positions 85, 138, and 178 replaced with other amino acid residue(s). Most preferably, the said variant has all the cysteine residues in positions 85, 138, and 178 replaced with serine residues (see Tudyka & Skerra, 1997 and SEQ ID NO: 5).

In addition, the said recombinant construct should comprise a nucleotide sequence encoding an N-terminal signal peptide, which allows for secretion of the said fusion protein from a host cell into the culture medium. For production of a human protein such as SSAO in a eukaryotic cell a homologous signal peptide is preferred. For production of SSAO in HEK293 cells e.g. a mouse IgG1 heavy chain signal peptide (Kabat et al., 1991) may be used. Other suitable signal peptides are known in the art and are described in e.g. Kabat et al., supra.

Several methods have been described for site-specific cleavage of fusion proteins based on treatment with chemical agents such as CNBr or hydroxylamine, or enzymes such as enterokinases, Factor Xa, thrombin, subtilisin or other proteases (see e.g. Nilsson et al. (1997) and references therein). According to this invention, the said fusion partner can conveniently be removed from human SSAO by protease cleavage. The protease to be used for cleavage can e.g. be a 3C protease from the picornavirus family, e.g. a rhinovirus or enterovirus 3C protease (Walker et al., 1994). Consequently, the protease cleavage site can preferably be a cleavage site for a 3C-protease from the picornavirus family, e.g. a rhinovirus or enterovirus 3C protease. In one exemplified form of the invention, the said 3C protease cleavage site comprises the amino acid sequence EALFQG (SEQ ID NO: 6). However, the skilled person will be able to identify other suitable cleavage sites, see e.g. Blom et al. (1996) and references therein.

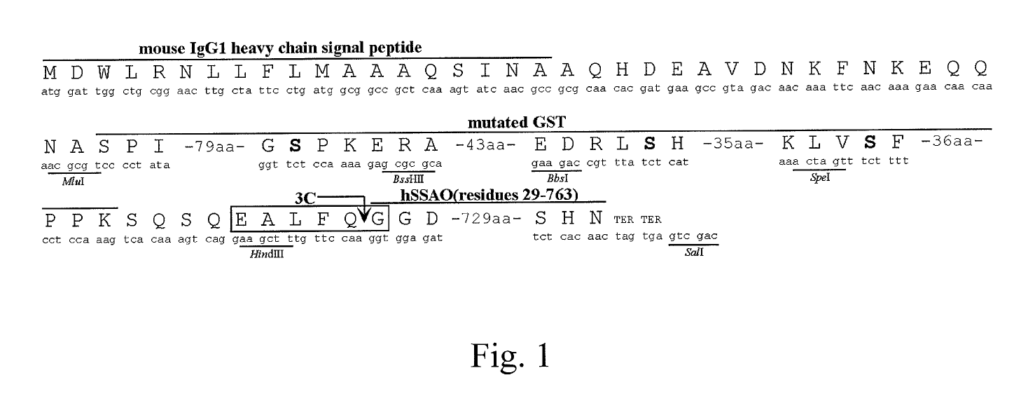

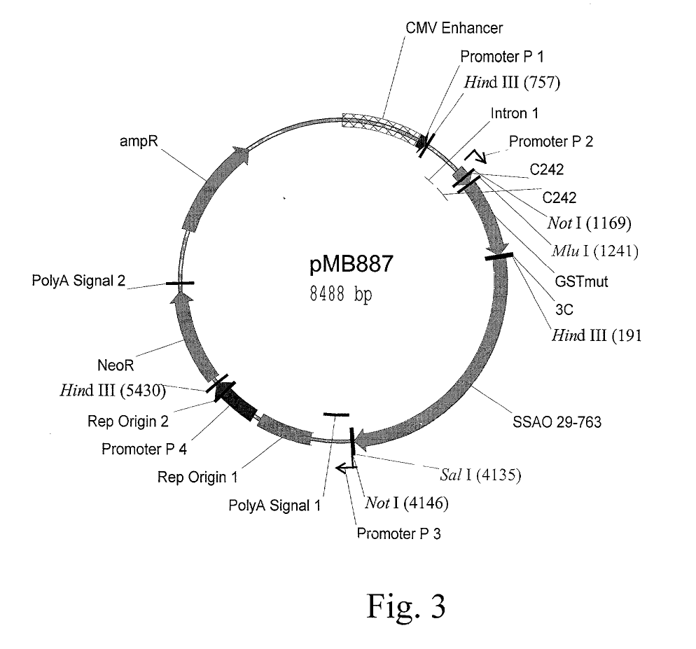

The recombinant construct according to the invention could e.g. comprise a nucleotide sequence encoding essentially the amino acid sequence shown in FIG. 1. The invention also provides an expression vector, prepared according to standard methods, comprising the recombinant construct according to the invention. Such an expression vector is exemplified by the expression vector termed pMB887, shown in FIG. 3.

In another aspect, the invention provides a method for the purification of a recombinant human SSAO polypeptide comprising the steps of:

(i) transfecting cells with an expression vector according to the invention, as defined above;

(ii) culturing the said cells under conditions allowing for the fusion protein expressed by the vector to be secreted into the cell medium;

(iii) binding the obtained fusion protein to a medium comprising a ligand having affinity for the fusion partner;

(iv) separating the said fusion partner and the SSAO polypeptide; and

(v) recovering the purified human SSAO polypeptide.

The fusion partner can be separated from the human SSAO variant either when the fusion protein is still attached to the affinity ligand, or when the fusion protein has been released from the affinity ligand. When the said fusion partner is GST, the said ligand having affinity for the fusion partner is preferably glutathione, or a derivative thereof. Alternatively, antibodies directed to GST could be used as affinity ligands.

As mentioned above, the fusion partner can be separated from human SSAO by protease cleavage with e.g. a picornavirus, such as rhinovirus, 3C-protease. The said protease can be fused to a fusion partner, thereby obtaining a “fusion protease” (see Walker et al., 1994; Gräslund et al., 1997). Such a fusion partner can conveniently be the same fusion partner as used for the SSAO polypeptide, e.g. glutathione S-transferase. However, other suitable fusion partners for proteases, such as albumin-binding protein from streptococcal Protein G, are known in the art, see e.g. Gräslund et al., 1997. The said fusion protease can be separated from the SSAO polypeptide by a process comprising binding the fusion protease to a medium comprising a ligand having affinity for the said fusion partner. Consequently, when the fusion partner is GST, the said ligand is preferably glutathione, or a derivative thereof. As mentioned above, antibodies directed to the fusion partner could also be used as affinity ligands. A commercially available system is the PreScission Protease (Amersham Pharmacia Biotech,) which is a genetically engineered fusion protein consisting of S. japonicum GST and human rhinovirus 3C protease.

For certain application, it might be advantageous to have SSAO immobilized. This may be achieved e.g. by an affinity-tag such as GST as described above. Examples of applications where a fusion protein is immobilized via an affinity-tag include: capture of protein ligands, analysis of protein-protein interactions, and use in bioreactors (Nilsson et al., 1996; Nord et al., 1997; Shpigel et al., 1999). However, many alternative methods for protein immobilization are described (see e.g. Tischer and Kasche, 1999, and references therein), that also may be applicable for immobilization of GST-SSAO or SSAO after removal of the fusion partner, such as covalent binding and non-covalent adsorption. In addition, the SSAO protein might also be encapsulated in e.g. sol-gel or an artificial cell e.g. a liposome (see e.g. Liang et al., 2000, and references therein).

One advantage with an affinity-tag such as GST is that an oriented immobilization can be achieved, often in a one-step procedure directly from e.g. a cell lysate (Nilsson et al., 1997; Saleemuddin, 1999). This may result in good steric accessibility of active binding sites and increased stability (Saleemuddin, 1999; Turkova, 1999). Examples of alternative affinity-tag approaches that has been used for immobilization of proteins are e.g. peptides and proteins that can be specifically biotinylated by biotin ligase and used as fusion partners to take advantage of the very strong interaction (K_{d}˜10⁻¹⁵) between biotin and streptavidin or avidin (Nilsson et al., 1997), and CBDs which binds specifically to cellulose (Linder et al., 1998; Tomme et al., 1998). Oriented immobilization of a protein may also be achieved by using immobilized antibodies that binds the protein or through carbohydrate moieties that may be present on the protein surface (Turkova, 1999).

Recently, amine oxidase from pea seedlings was immobilized using a modified carbon paste to yield a biosensor for determination of biogenic and synthetic amines (Wimmerova and Macholan, 1999). Similarly, recombinant human SSAO might be immobilized for construction of biosensors to detect e.g. the cardiovascular toxin allylamine which is used in industrial organic processes and is a substrate for SSAO (Boor and Hysmith, 1987; Conklin et al., 1998). When immobilized, recombinant SSAO may be envisioned to mimic a membrane-anchored SSAO and its characteristics, which might differ from the soluble state.

Consequently, as shown in the following examples, the invention provides a procedure for the production of a highly purified soluble recombinant human SSAO with enzymatic activity. The exemplified procedure involves the use of a mutant form of S. japonicum glutathione S-transferase (GST), designed for transport out of the host cells (Tudyka and Skerra, 1997), as an affinity fusion partner. The fusion protein was secreted from mammalian cells and could be purified directly from the culture medium by glutathione-affinity chromatography. By specific proteolysis and an additional glutathione-affinity chromatography step, the fusion partner and the protease were removed, whereby pure, soluble and highly active recombinant human SSAO protein was obtained in milligram quantities. To the inventors' knowledge, this is the first time an active recombinant soluble form of the human SSAO protein has been produced and purified to near homogeneity.

It is believed that the disclosed process for production of recombinant human SSAO will be applicable also to other mammalian amine oxidases, such as the human placenta diamine oxidase (Zhang et al., 1995) and the human retina-specific amine oxidase (Imamura et al., 1998), as well as for other secretable proteins. The disclosed process may also facilitate the discovery and identification of modifications e.g. the identification of the active site cofactor, e.g. by isolation of cofactor-containing peptides or by crystal structure determination.

In the following examples, it is shown that SSAO is active and soluble without its transmembrane region, and that GST can be proteolytically removed. These findings support the hypothesis that SSAO is released into circulation by proteolytic cleavage near the transmembrane region (shedding), a process which is common for Type I and Type II membrane proteins (Hooper et al., 1997). The elevated SSAO activity in plasma in e.g. diabetes (Boomsma et al., 1999) may thus be the consequence of increased proteolytic activity of a protease that cleave the membrane-anchored SSAO, or of increased surface localization increasing the substrate availability for an existing protease.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### EXPERIMENTAL METHODS

PCR-amplification and Cloning of the Human SSAO Gene from Aorta cDNA

Two PCR-primers were designed with the help of the published cDNA sequence of human placenta amine oxidase (GenBank Accession No. U39447; Zhang and McIntire, 1996). The 5′-primer XNQZ-15 (5′-CCG GAA TTC CAA CGC GTC CAT GAA CCA GAA GAC AAT CCT CGT G-3′; SEQ ID NO: 7) was designed to hybridize to the 5′-end of the SSAO coding sequence including the ATG start codon and to contain the restriction enzyme cleavage sites EcoRI and MluI for cloning. The 3′-primer XNQZ-17 (5′-CCC CCA AGC TTG TCG ACT CAC TAG TTG TGA GAG AGA AGC CCC CCC-3′; SEQ ID NO: 8) was designed to hybridize to the 3′-end including the native stop codon TAG followed by an additional stop codon TGA and two restriction enzyme cleavage sites for cloning, SalI and HindIII. As template for the PCR 0.5 μl human aorta or human smooth muscle cell QUICK-Clone cDNAs (1 ng/μl, Clontech Laboratories, Palo Alto, Calif.) were tested. The following conditions was used for the PCR-reaction, 20 pmol of each primer XNQX-15 and XNQZ-17, 1 μl dNTPs (10 mM), 1 μl Advantage cDNA Polymerase Mix (Clontech), 5 μl 10×cDNA PCR reaction buffer (Clontech) in a total volume of 50 μl. Amplification was performed with a Perkin-Elmer 2400 thermocycler (Perkin-Elmer, Norwalk, Conn.). The PCR-program consisted of an initial denaturation at 94° C. for 5 min, 35 cycles of 94° C. for 30 s, 60° C. for 30 s and 72° C. for 3 min followed by a final extension at 72° C. for 3 min. TA-cloning was then used to insert the PCR-product into the vector pCR2. 1-TOPO (Invitrogen, Carlsbad, Calif.). The cloned PCR-fragment was sequenced in both directions according to a standard protocol for dye terminator cycle sequencing and analyzed on a DNA sequencer ABI 377 (Applied Biosystems, Foster City, Calif.).

Construction of Vectors for Expression of SSAO in Mammalian Cells

A vector for expression of the complete SSAO protein in mammalian cells was prepared by insertion of the EcoRI and SalI fragment from the pCR2.1 ITOPO-SSAO vector into the same sites of the vector pCI-neo (Promega, Madison, Wis.), resulting in the vector pMB843. This vector was used as template for PCR-amplification of the region corresponding to residues 29-763 of the human SSAO (Zhang and McIntire, 1996). A 5′-primer 5′-GAG GAA GCT TTG TTC CAA GGT GGA GAT GGG GGT GAA-3' (SEQ ID NO: 9) was synthesized containing codons for a partial 3C protease cleavage site (see below) and a HindIII restriction enzyme cleavage site upstream of the codon for residue 29. The 3′-primer 5′-GCA TTC TAG TTG TGG TTT GTC-3' (SEQ ID NO: 10) is a pCI-neo vector specific primer annealing downstream of the cloned SSAO fragment. The resulting PCR-product was digested with HindIII and NotI and cloned into the plasmid pET38b(+) (Novagen, Inc., Madison, Wis.) cut with same enzymes, resulting in pET38-SSAO. DNA sequencing was performed as described above to verify expected sequence of the cloned SSAO fragment.

A mutated form (SEQ ID NO: 5) of the glutathione S-transferase (GST) from S. japonicum previously used as a secretable enzymatically active dimerization module for a recombinant protein (Tudyka and Skerra, 1997) was prepared by PCR-mediated mutagenesis and assembly of fragments as described below. The mutations was performed to replace three cysteine residues 85, 138, and 178 located close to the GST protein surface as revealed in the crystal structure of the S. japonicum GST (Lim et al., 1994; Tudyka and Skerra, 1997) with serine residues in order to avoid unwanted disulphide formation after export of the GST fusion protein to an oxidizing environment (Tudyka and Skerra, 1997). The following PCR-primers were used to construct the mutated GST and to introduce the first part of a 3C protease cleavage site (see below) as well as suitable restriction enzyme cleavage sites for cloning. In addition, the primers introduce internal restriction sites for control cleavage and for possibility to assemble PCR-fragments by ligation: ROEL-1 (5′-GCC GGA ATT CGA CGC GTC CCC TAT ACT AGG TTA TTG G-3′; SEQ ID NO: 11) contains EcoRI and MluI for cloning and anneals to codons 2-8 of GST (M14654); ROEL-2 (5′-CTC TGC GCG CTC TTT TGG AGA ACC CAA CAT GTT GTG C-3′; SEQ ID NO: 12) contains a BssHII site; ROEL-3 (5′-GGT TCT CCA AAA GAG CGC GCA GAG ATT TCA ATG CTT GAA G-3′; SEQ ID NO: 13) contains a BssHII site; ROEL-4 (5′-ATG AGA TAA ACG GTC TTC GAA CAT TTT CAG CAT TTC-3′; SEQ ID NO: 14) contains a BbsI site; ROEL-5 (5′-GTT CGA AGA CCG TTT ATC TCA TAA AAC ATA TTT AAA TGG TGA TC-3′; SEQ ID NO: 15) contains a BbsI site; ROEL-6 (5′-AAA AGA AAC TAG TTT TGG GAA CGC ATC CAG GCA-3′; SEQ ID NO: 16) contains a Spel site; ROEL-7 (5′-CCC AAA ACT AGT TTC TTT TAA AAA ACG TAT TGA AGC TAT C-3′; SEQ ID NO: 17) contains a Spel site; ROEL-8 (5′-ACC CAA GCT TCC TGA CTT TGT GAC TTT GGA GGA TGG TCG CCA CC-3′; SEQ ID NO: 18) contains HindIII for cloning and anneals to codons 212-218 of GST (M14654). ROEL-8 will also introduce codons for a spacer-sequence SQSQ before a partial 3C protease cleavage site. Overlapping parts of the GST gene were amplified in separate PCR-reactions with primer pairs ROEL-1/2, ROEL-3/4, ROEL-4/5 and ROEL-7/8, using plasmid pGEX-6P-2 (Amersham Pharmacia Biotech) as template. This allowed the complete mutated GST gene to be assembled by mixing the four PCR-fragments and using them as templates in a PCR reaction with primers ROEL-1 and ROEL-8. The PCR-reactions was performed using the Advantage cDNA PCR Kit (Clontech). In the next step the GST fragment was digested with EcoRI and HindIII and cloned into the same sites of pUC 18 (Amersham Pharmacia Biotech), yielding pMB809. DNA sequencing was performed as described above to confirm the expected sequence of the mutated GST fragment. The pMB809 vector was cleaved with EcoRI and HindIII and the GST fragment was isolated and cloned upstream of the SSAO fragment in the pET38-SSAO vector cut with the same enzymes. This step resulted in the creation of a complete 3C protease cleavage site EALFQG (SEQ ID NO: 6) of human rhinovirus-14 and coxsackievirus (Miyashita et al., 1996; Wang et al., 1997) between GST and SSAO (residues 29-763) (see FIG. 1).

The GST-SSAO fragment was cloned in the MluI and SalI site of the vector pMB565, in which a mutated signal sequence of a murine IgG1 heavy chain (FIG. 1) is cloned in the multilinker of the mammalian expression vector pCI-neo (Promega). The resulting GST-SSAO expression vector was named pMB887 (FIG. 3).

Transfection and Selection of Stable Clones

Three 25 cm² T-flasks were seeded with approximately 4×10⁵ human embryo kidney 293 cells (HEK293 cells, ATCC CRL-1573, Rockville, Md.). Cells were grown to ˜50% conflueny in growth medium containing Dulbecco's modified Eagle's medium (DMEM) supplemented with 10% fetal bovine serum (FBS) and 2 mM L-Glutamine. The FBS was heat-inactivated at 56° C. for 30 min before mixed with the growth medium components. The expression vector pMB887 was then introduced into the cells by liposome-mediated transfection using LipofectAMINE according to the manufacturer's recommendations (Life Technologies, Frederick, Md.). After 48 hours of growth the medium was changed in all flasks to growth medium supplemented with 1 mg/ml geneticin (G418) for selection of stably transfected cells. Approximately two weeks later, resistant cells emerged and were grown to confluency. Cells from the three T-flasks were pooled (clone-mixture) and diluted in growth medium supplemented with 1.2 mg/ml G418 and seeded in a 15-cm Petri dish. Individual colonies emerged after two weeks and were picked to be expanded individually for subsequent analysis of GST-SSAO production. Detection of GST proteins in collected medium from expanded clones was performed using the GST 96-Well Detection Module (Amersham Pharmacia Biotech). Seven positive clones were selected and frozen.

Production of GST-SSAO in Cell Factories

Clone number 10 was expanded and cultured in growth medium containing DMEM supplemented with 5% FBS (heat-inactivated), 2 mM L-Glutamine and 1.2 mg/ml G418 and used to seed a 6320 cm²Nunc Cell Factory (Nalge Nunc Int., Naperville, Ill.) containing 1500 ml of growth medium and grown at 37° C. After four days of growth, cells were confluent and medium was collected. New growth medium (1500 ml) with reduced amount of FBS (2%) was then added to the cells in the same Cell Factory followed by harvest of conditioned medium after three days. This procedure was repeated once resulting in a total of ˜4.5 liters of harvested medium from one Cell Factory. Collected medium was centrifuged and stored at −70° C.

Concentration of Conditioned Cell Medium

Frozen conditioned medium from two Cell Factories (9.4 liters) was thawed in a water-bath at 30° C. The material was pumped through an Omega membrane (MWCO (Molecular-Weight Cut-Off) 10000) using a Centramate ultra-filtration equipment (Pall Filtron, Northborough, Mass.), until a volume of 600 ml was achieved. The retentate was filtered through a 0.45 μm filter, Sartobran P, equipped with a 0.65 μm prefilter (Sartorius, Göttingen, Germany). Remaining filtrate in tubings was displaced by 250 ml of phosphate-buffered saline (PBS) yielding 850 ml of filtered sample.

Purification and Cleavage of GST-SSAO

The GST-SSAO fusion protein was purified by glutathione-affinity chromatography on a HR 10/10 column (Amersham Pharmacia Biotech) packed with 8 ml glutathione-Sepharose 4 Fast Flow (Binds˜10 mg GST/ml gel, Amersham Pharmacia Biotech), equilibrated with 10 column volumes of PBS. The filtered material (850 ml), was loaded at 0.9 ml/min over night at room temperature. Flow-through material was collected for analysis and stored at −20° C. After washing the column with PBS, bound proteins were eluted with elution buffer (20 mM GSH, 0.1 M NaCl, 0.1 M Tris-HCl, pH 8.2).

The eluate was loaded on a HiPrep Desalt 26/10 column (Amersham Pharmacia Biotech) equilibrated with helium-sparged cleavage-buffer (150 mM NaCl, 1 mM EDTA, 50 mM Tris-HCl, pH 7.5 at 25° C.) and the protein peak was collected. Cleavage was started by adding DTT (dithiothreitol) to 5 mM and 380 units of PreScission protease (Amersham Pharmacia Biotech). The PreScission Protease is a genetically engineered fusion protein consisting of GST and human rhinovirus 3C protease and cleaves specifically between the Gln (Q) and Gly (G) residues of its recognition sequence.

The cleavage mixture was incubated at 5° C. After 63 hours of incubation the material was loaded on a glutathione-Sepharose column as described above, equilibrated with cleavage buffer. The flow-through (36 ml) was collected and stored at 5° C. for approximately one week in an open tube. Samples were withdrawn and analyzed by SDS-PAGE (non-reducing). Proteins captured on the column were eluted with elution buffer for analysis. The collected protein sample was applied on a JumboSep device (MWCO 30000, Pall Filtron) for buffer exchange and concentration. Fiva cycles of centrifugation and dilution with a buffer containing 50 mM Tris-HCl (pH 7.5) and 150 mM NaCl were performed. A sample was taken for different analyses. The buffer exchanged and concentrated material (4.2 ml) was then stored at −70° C.

Protein Analyses

The purification and size of SSAO were analyzed by SDS-PAGE. Samples were electrophoresed in the presence or absence of 2-mercaptoethanol in gradient gels 4-20% or 4-12% (Novex, Copenhagen, Denmark) and proteins were visualized by Coomassie staining (PhastGel Blue R, Amersham Pharmacia Biotech). Protein concentrations were determined with Coomassie Plus protein assay reagent kit (Pierce, Rockford, Ill.) in 96-well plates with bovine serum albumin as standard according to the manufacturer's procedure.

Size exclusion chromatography was performed on a Superdex 200 PC 3.2/30 column (Amersham Pharmacia Biotech) using the SMART System (Amersham Pharmacia Biotech). The column was equilibrated at room temperature with a buffer containing 50 mM Tris-HCl (pH 7.5), 150 mM NaCl and 1 mM EDTA. Injection volume was 10 μl and samples were eluted at a flow rate of 0.1 ml/min. For column calibration molecular weight markers Blue Dextran 2000 (˜2000 kDa), thyroglobulin (669 kDa), ferritin (440 kDa), catalase (232 kDa) and aldolase (158 kDa) from the Gel Filtration HMW Calibration Kit (Amersham Pharmacia Biotech) was used.

N-terminal sequencing was performed on purified GST-SSAO and SSAO by repeated Edman degradation using a HP G1000A protein sequencer coupled to a HP 1090 PTH analyzer (Hewlett Packard, Palo Alto, Calif.). The GST-SSAO sample was desalted to remove glutathione prior to analysis. SSAO was taken from the flow-through of the glutathione-Sepharose column after cleavage.

A spectrophotometric assay for monoamine oxidases described by Holt and coworkers (Holt et al., 1997) was used to determine amine oxidase activity in samples from the different purification steps. The assay was performed in 96-well microtiter plates incubated at 37° C. in a SPECTRAmax 250 microplate spectrophotometer (Molecular Devices, Sunnyvale, Calif.). The reagent mix containing 1 mM vanillic acid (Sigma, St. Louis, Mo.), 500 μM 4-aminoantipyrine (Sigma), and 4U ml⁻¹ peroxidase (type VI from horseradish, Sigma) in 0.2 M potassium phosphate buffer (pH 7.6) was prepared on the same day assays were performed and kept at 5° C. until used. Reactions were started by mixing 50 μl sample, 50 μl reagent mix and 200 μl potassium phosphate buffer with or without 750 μM benzylamine hydrochloride (Sigma) and were performed in triplicate. In order to obtain blank reference values, wells were analyzed with buffer added in place of sample. Absorbance changes were followed at 490 nm for 10-40 minutes. Standard curves were prepared with dilutions of a stock solution of H₂O₂ in potassium phosphate buffer ranging from 10 nmol/well to 120 nmol/well. When inhibition experiments were carried out, the samples were incubated in 300 μM semicarbazide at 37° C. for 30 minutes, before addition of the benzylamine solution.

The invention will now be described with reference to the following examples. These are only intended to exemplify the invention and are not to be considered as limiting the scope of the invention in any way.

### EXAMPLES

### Example 1

Cloning of SSAO cDNA

A PCR-strategy was used to amplify the gene of a human SSAO from human aorta cDNA. The PCR-primers were designed to include sequences flanking the human placenta amine oxidase gene (Zhang and McIntire, 1996) and to include restriction enzyme cleavage sites for cloning into different expression vectors. The ˜2300 bp PCR-product was cloned and subsequent DNA-sequencing showed that the sequence of the cloned PCR-product was identical to the human placenta amine oxidase sequence (Zhang and McIntire, 1996) and to the VAP-1 sequence cloned from lung cDNA (Smith et al., 1998).

### Example 2

### Purification of Membrane-bound SSAO (Example for Comparison)

Attempts to produce a recombinant SSAO protein showed that active SSAO could be produced in human embryo kidney (HEK293) cells using the pMB843 vector in which the entire coding sequence of human SSAO was cloned. Active protein was found after extraction using solubilizing agents, but only microgram amounts of protein could be partially purified.

### Example 3

Rationale and Design of a Gene Construct for Expression of a Soluble Form of SSAO

An alternative strategy was developed for production of a non-membrane-bound SSAO in mammalian cells. Purification and detection were performed by replacing the N-terminal region containing the putative membrane spanning peptide with an affinity fusion partner having an inherent dimerization propensity. The strategy also involved the use of a secretable affinity fusion partner to be able to secrete the fusion protein into the culture medium. A mutated variant of S. japonicum glutathione S-transferase (GST) was selected. This mutant GST retains its activity as well as its propensity to dimerize and have been optimized for secretion (Tudyka and Skerra, 1997).

A protease cleavage site was designed to enable release of SSAO from the purified GST-SSAO fusion protein. Scanning of the predicted amino acid sequence revealed an arginine at position 28 flanked by three glycine residues. Several human proteases cleave after basic residues (Carter, 1990; Hooper et al., 1997) and short stretches of glycine residues have been suggested to enhance accessibility to proteases (Carter, 1990). In addition, the proteolytic release of the extracellular region (shedding) of many membrane-anchored proteins into the blood stream occurs close to the membrane (Hooper et al., 1997). The glycine residue at position 29 was therefore chosen to be linked to a suitable substrate for site-specific proteolysis after purification of the GST-SSAO fusion protein. Thus, a protease that can cleave a substrate having a glycine in the P1' position and having high specificity was desired. Several commercial proteases exist having these two properties such as factor Xa, thrombin, enterokinase and 3C protease (Nilsson et al., 1997). The ability to easily capture the protease after cleavage was another factor considered, leading to the selection of a commercially available 3C protease fused to GST. The 3C protease cleavage site EALFQG (SEQ ID NO: 6) (Miyashita et al., 1996; Wang et al., 1997) was introduced in the GST-SSAO fusion construct (FIG. 1).

The GST-SSAO fragment was cloned in frame with a signal sequence to achieve secretion of the GST-SSAO fusion protein into the culture medium. A signal sequence derived from the heavy chain of a murine antibody was used (see FIG. 1). The final construct thus encoded a fusion protein comprising of an antibody signal peptide, an 18 amino acid spacer region, the mutated GST protein, a substrate sequence for the 3C protease and residues 29-763 of the human SSAO protein cloned from human aorta cDNA (FIG. 1). The calculated molecular weight of the unmodified GST-SSAO fusion protein is 112 kDa.

### Example 4

Initial Analyses on Conditioned Medium from HEK293 Cells Transfected with the GST-SSAO Expression Vector

Benzylamine oxidase activity in the conditioned medium from small-scale cultures of HEK293 cells, stably transfected with the GST-SSAO expression vector pMB887, indicated that GST-SSAO was secreted into the culture medium. Further analyses showed that glutathione-Sepharose beads could be used to purify small amounts of the GST-SSAO fusion protein directly from the conditioned medium (data not shown), and that the purified material had benzylamine oxidase activity. Interestingly, the GST-SSAO fusion protein was found to be active also when immobilized on the glutathione Sepharose beads. The amount of GST-SSAO fusion protein in the conditioned medium was calculated to be ˜1 mg/l, by estimation of the amount of protein captured on the beads.

### Example 5

Preparative Purification and Site-specific Cleavage of the GST-SSAO Fusion Protein

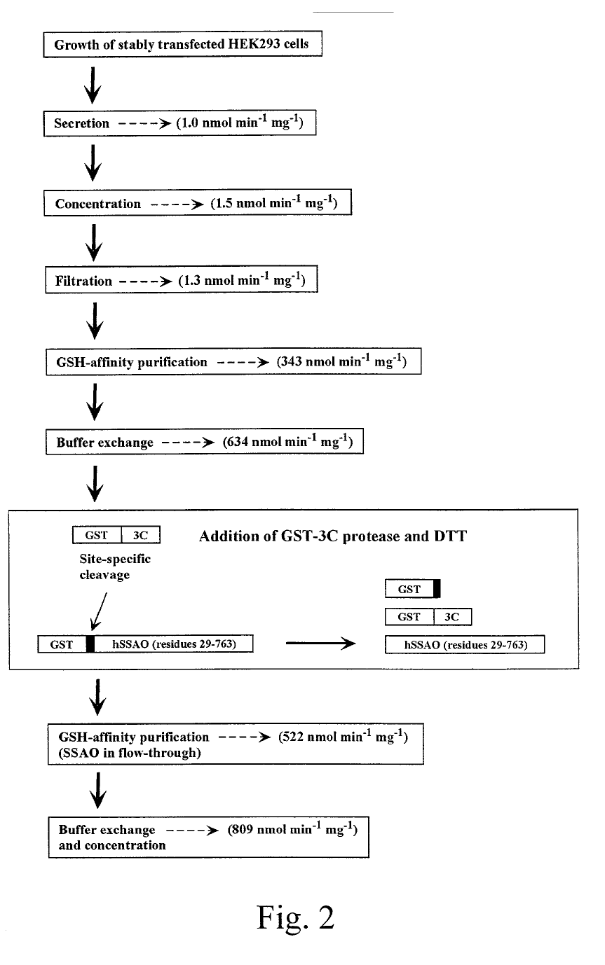

An overview of the affinity purification based procedure is shown in FIG. 2. The results of the purification are summarized in Table 1. One selected clone was expanded and grown in Cell Factories to generate larger amounts of GST-SSAO for purification. The harvested conditioned medium were concentrated and filtrated to reduce the time for loading on the glutathione-Sepharose column. Glutathione-affinity chromatography was then applied to purify the GST-SSAO fusion protein from the concentrated and filtered conditioned medium. Proteins captured on the column were eluted with 20 mM GSH and analyzed by SDS-PAGE under reducing conditions. This showed that the GST-SSAO fusion protein had high purity and that it could be isolated from large amounts of other proteins in the culture medium in a single step. The GST-SSAO fusion protein migrated in level with the 116 kDa protein in the molecular weight marker. In total 8.8 mg of protein was recovered from the glutathione-Sepharose column. The specific activity of the GST-SSAO fusion protein was determined to 343 nmol·min⁻¹·mg⁻¹. Interestingly, the specific activity was almost doubled (634 nmol·min⁻¹·mg⁻¹) by the buffer exchange step which removed the reducing agent GSH.

The glutathione-affinity purified GST-SSAO was cleaved with the GST-3C protease fusion protein (46 kDa) to remove the GST fusion partner from SSAO. Analytical experiments suggested that cleavage was slow, but precise, with no observable side-products. Moreover, complete cleavage could be obtained after ˜48 hours incubation. The cleavage mixture was passed over the glutathione-Sepharose column to capture the removed GST fusion partner and the GST-3C protease. Flow-through material was collected and analyzed by SDS-PAGE under reducing conditions which showed only the expected SSAO product with a molecular weight of ˜97 kDa. Captured material was also analyzed, which showed only the GST fusion partner (˜28 kDa) and the GST-3C protease. This indicated that a complete cleavage had occurred and all GST containing proteins had been captured on the glutathione-Sepharose column. It also indicated that all SSAO protein had passed through the column since no SSAO protein was seen in the eluted material.

The specific activity of the purified SSAO protein was determined to 522 nmol·min⁻¹·mg⁻¹ which was less than the specific activity determined before cleavage. Since DTT had been used to ensure 3C protease activity during cleavage of the GST-SSAO fusion protein, we made an SDS-PAGE analysis (non-reducing) to see if the cleavage buffer had affected possible disulphide bridges in the SSAO homodimer (Kurkijärvi et al., 1998; Smith et al., 1998; Salminen et al., 1998). Only presumed SSAO monomers (˜97 kDa) could be seen (data not shown). However, the SSAO protein was transformed to ˜170 kDa in size (analyzed by SDS-PAGE) during storage at 5° C., indicating that one or several disulphides were formed. The cleavage buffer was removed by diafiltration and SDS-PAGE analysis showed that the SSAO protein was still apparently dimeric with a molecular weight of ˜170 kDa. In total 3.6 mg of recombinant SSAO was obtained from 9.4 liters of conditioned medium having a specific activity of 809 nmol·min⁻¹·mg⁻¹. The overall yield in the process was 22% based on determined benzylamine oxidase activity.

Interestingly, the GST fusion partner did not significantly affect the benzylamine oxidase activity of the SSAO protein. The specific activity of the purified GST-SSAO fusion protein after the buffer exchange step, was determined to 634 nmol·min⁻¹·mg⁻¹. After removal of the GST fusion partner, the specific activity of SSAO was determined to 809 nmol·min⁻¹·mg⁻¹. However, the molecular mass of the GST fusion partner is ˜25% of the GST-SSAO fusion protein and the increase in specific activity after removal of GST was in the same range. This opens up possibilities to use the fusion protein for enzyme characterization. Furthermore, an affinity fusion partner such as GST can be used to bind or immobilize a recombinant protein in a directed manner on solid supports to study e.g. protein-protein interactions and enzyme characteristics (Nilsson et al., 1997). The GST-SSAO fusion protein was indeed active when it was bound to glutathione-Sepharose beads.

### Example 6

### Initial Characterization of Purified SSAO Proteins

A gel filtration experiment was performed to analyze the size of the SSAO protein under non-denaturing conditions. A sample from the SSAO protein material that migrated as a dimeric protein when investigated by SDS-PAGE under non-reducing conditions was loaded on a calibrated analytical Superdex 200 column. The SSAO protein eluted at 1.29 ml, which was slightly faster than catalase (232 kDa), which eluted at 1.35 ml.

N-terminal amino acid sequencing of the purified SSAO protein showed that the GST-3C protease had specifically cleaved the 3C protease substrate sequence EALFQG (SEQ ID NO: 6) in the GST-SSAO fusion protein (FIG. 1). Twenty-nine amino acids were determined and corresponded exactly to residues number 29-58 in the predicted SSAO amino acid sequence (SEQ ID NO: 2). N-terminal sequencing was also performed on the GST-SSAO fusion protein, which showed that the signal peptide had been processed as anticipated.

Finally, the purified SSAO protein was found to be sensitive to inhibition by semicarbazide as expected. In the presence of 0.1 mM semicarbazide more than 95% of the benzylamine oxidase activity was inhibited.

TABLE I
Purification of recombinant human SSAO

| Purification step | Total volume | Total protein | Total SSAO activity^{a} | Specific activity | Yield |
|---|---|---|---|---|---|
| (sample) | (ml) | (mg) | (nmol min⁻¹) | (nmol min⁻¹ mg⁻¹) | (%) |
| Conditioned medium | 9400 | 9024 | 9243 | 1.0 | — |
| Concentrated medium | 600 | 9060 | 13200 | 1.5 | 100 |
| Filtrate | 850 | 9065 | 11900 | 1.3 | 90 |
| GSH-affinity step-1 (eluate) | 6.8 | 8.8 | 3029 | 343 | 23 |
| Buffer exchange | 15 | 8.9 | 5624^{b} | 634^{b} | 43 |
| GSH-affinity step-2 (flow-through)^{c} | 36 | 5.5 | 2859 | 522 | 22 |
| Diafiltrate^{d} | 4.2 | 3.6 | 2919 | 809 | 22 |

^{a}Activity was measured as nmol of H₂O₂ produced per minute using 0.5 mM benzylamine as substrate. SSAO activity was confirmed with 0.1 mM semicarbazide as inhibitor.§
^{b}Measured before addition of 5 mM DTT and GST-3C protease.
^{c}This step was performed after site-specific cleavage of the GST-SSAO fusion protein to capture removed GST fusion partner and GST-3C protease.
^{d}Buffer exchange and sample concentration by ultra-filtration.

References

Blom, N., Hansen, J., Blaas, D. and Brunak, S. (1996) Cleavage site analysis in picornaviral polyproteins: Discovering cellular targets by neural networks. Protein Science 5, 2203-2216.

Boomsma, F., van Veldhuisen, D. J., de Kam, P. J., Man in't Veld, A. J., Mosterd, A., Lie, K. I., and Schalekamp, M. A. (1997) Plasma semicarbazide-sensitive amine oxidase is elevated in patients with congestive heart failure. Cardiovasc. Res. 33(2), 387-391.

Boomsma, F., van den Meiracker, A. H., Winkel, S., Aanstoot, H. J., Batstra, M. R., Man in't Veld, A. J., and Bruining, G. J. (1999) Circulating semicarbazide-sensitive amine oxidase is raised both in type I (insulin-dependent), in type II (non-insulin-dependent) diabetes mellitus and even in childhood type I diabetes at first clinical diagnosis. Diabetologia 42(2), 233-237.

Boor, P. J., and Hysmith, R. M. (1987) Allylamine cardiovascular toxicity. Toxicology 44(2), 129-145.

Buffoni, F. (1993) Properties, distribution and physiological role of semicarbazide-sensitive amine oxidases. Curr. Top. Pharmacol. 2, 33-49.

Callingham, B. A., Crosbie, A. E., and Rous, B. A. (1995) Some aspects of the pathophysiology of semicarbazide-sensitive amine oxidase enzymes. Prog. Brain Res. 106, 305-321.

Carter, P. (1990) Site-specific proteolysis of fusion proteins. In Protein purification: From molecular mechanism to large-scale processes (Am. Chem. Soc. Symp. Ser. No. 427) (Ladish, M. R., Willson, R. C., Painton, C. C., and Builder, S. E., eds), pp. 181-193, American Chemical Society Press.

Castillo, V., Lizcano, J. M., Visa J., and Unzeta, M. (1998) Semicarbazide-sensitive amine oxidase (SSAO) from human and bovine cerebrovascular tissues: biochemical and immunohistological characterization. Neurochem. Int. 33(5), 415-423.

Conklin, D. J., Langford, S. D., and Boor, P. J. (1998) Contribution of serum and cellular semicarbazide-sensitive amine oxidase to amine metabolism and cardiovascular toxicity. Toxicol. Sci. 46(2), 386-392.

Dwyer, M. A., Huang, A. J., Pan, C. Q., and Lazarus, R. A. Expression and characterization of a DNase I-Fc fusion enzyme. (1999) J. Biol. Chem. 274(14), 9738-9743.

Ekblom, J. (1998) Potential therapeutic value of drugs inhibiting semicarbazide-sensitive amine oxidase: vascular cytoprotection in diabetes mellitus. Pharmacol. Res. 37(2), 87-92.

Gräslund, T., Nilsson, J., Lindberg, A. M., Uhlén, M., and Nygren, P. Å. (1997) Production of a thermostable DNA polymerase by site-specific cleavage of a heat-eluted affinity fusion protein. Protein Expression Purif 9(1), 125-132.

Hartmann, C., and McIntire, W. S. (1997) Amine-oxidizing quinoproteins. Methods Enzymol. 280, 98-150.

Hollenbaugh, D., Chalupny, N. J., and Aruffo, A. (1992) Recombinant globulins: novel research tools and possible pharmaceuticals. Curr. Opin Immunol. 4(2), 216-219.

Holt, A., Sharman, D. F., Baker, G. B., and Palcic, M. M. (1997) A continuous spectrophotometric assay for monoamine oxidase and related enzymes in tissue homogenates. Anal. Biochem. 244(2), 384-392.

Holt, A., Alton, G., Scaman, C. H., Loppnow, G. R., Szpacenko, A., Svendsen, I., and Palcic, M. M. (1998) Identification of the quinone cofactor in mammalian semicarbazide-sensitive amine oxidase. Biochemistry 37(14), 4946-4957.

Hooper, N. M., Karran, E. H., and Turner, A. J. (1997) Membrane protein secretases. Biochem J. 321( Pt 2), 265-279.

Imamura, Y., Noda, S., Mashima, Y., Kudoh, J., Oguchi, Y., and Shimizu, N. (1998) Human retina-specific amine oxidase: genomic structure of the gene (AOC2), alternatively spliced variant, and mRNA expression in retina. Genomics 51(2), 293-298.

Jaakkola, K., Kaunismaki, K., Tohka, S., Yegutkin, G., Vanttinen, E., Havia, T., Pelliniemi, L. J., Virolainen, M., Jalkanen, S., and Salmi, M. (1999) Human vascular adhesion protein-1 in smooth muscle cells. Am. J. Pathol. 155(6), 1953-1965.

Kabat, E. A., Wu, T. T., Perry, H. M., Gottesman, K. S., and Foeller, C. (1991) Sequences of proteins of immunological interest. (U.S. Department of Health and Human Services; Public Health Service National Institutes of Health) NIH Publication No. 91-3242.

Kurkijärvi, R., Adams D. H., Leino R., Mottonen T., Jalkanen S., and Salmi, M. (1998) Circulating form of human vascular adhesion protein-1 (VAP-1): Increased serum levels in inflammatory liver diseases. J. Immunol. 161, 1549-1557.

Larsson, L. N., Johansson, C., Lindholm, L., and Holmgren, J. (1988) Mouse monoclonal antibodies for experimental immunotherapy promotes killing of tumor cells. Int. J. Cancer 42, 877-882.

Lewinsohn, R. (1984) Mammalian monoamine-oxidizing enzymes, with special reference to benzylamine oxidase in human tissues. Braz. J. Med. Biol. Res. 17(3-4), 223-256.

Liang, J. F., Li, Y. T., and Yang, V. C. (2000) Biomedical application of immobilized enzymes. J. Pharm. Sci. 89(8), 979-990.

Linder, M., Nevanen, T., Söderholm, L., Bengs, O., and Teeri, T. T. (1998) Improved immobilization of fusion proteins via cellulose-binding domains. Biotechnol. Bioeng. 60(5), 642-647.

Lim, K., Ho, J. X., Keeling, K., Gilliland, G. L., Ji, X., Ruker, F., and Carter, D. C. (1994) Three-dimensional structure of Schistosoma japonicum glutathion S-transferase fused with a six-amino acid conserved neutralizing epitope of gp41 from HIV. Protein Sci. 3(12), 2233-2244.

Lo, K. M., Sudo, Y., Chen, J., Li, Y., Lan, Y., Kong, S. M., Chen, L., An, Q., and Gillies, S. D. (1998) High level expression and secretion of Fc-X fusion proteins in mammalian cells. Protein Eng. 11(6), 495-500.

Lyles, G. A. (1996) Mammalian plasma and tissue-bound semicarbazide-sensitive amine oxidases: biochemical, pharmacological and toxicological aspects. Int. J Biochem. Cell Biol. 28(3), 259-274.

Lyles, G. A., and Pino, R. (1998) Properties and functions of tissue-bound semicarbazide-sensitive amine oxidases in isolated cell preparations and cell cultures. J. Neural. Transm. Suppl. 52, 239-250.

Meszaros, Z., Karadi, I., Csanyi, A., Szombathy, T., Romics, L., and Magyar, K. (1999) Determination of human serum semicarbazide-sensitive amine oxidase activity: a possible clinical marker of atherosclerosis. Eur. J. Drug Metab. Pharmacokinet. 24(4), 299-302.

Miyashita, K., Okunishi, J., Utsumi, R., Komano, T., Tamura, T., and Satoh, N. (1996) Cleavage specificity of coxsackievirus 3C proteinase for peptide substrate. Biosci. Biotechnol. Biochem. 60(4), 705-707.

Morris, N. J., Ducret, A., Aebersold, R., Ross, S. A., Keller, S. R., and Lienhard, G. E. (1997) Membrane amine oxidase cloning and identification as a major protein in the adipocyte plasma membrane. J. Biol. Chem. 272, 9388-9392.

Müller, K. M., Arndt, K. M., and Alber, T. (2000) Protein fusions to coiled-coil domains. Methods Enzymol. 328, 261-282.

Nakos, G., and Gossrau, R. (1994) Light microscopic visualization of semicarbazide-sensitive amine oxidase (benzylamine oxidase) using a cerium method. Folia Histochem. Cytobiol. 32(1), 3-10.

Nilsson, J., Larsson, M., Ståhl, S., Nygren, P. Å., and Uhlén, M. (1996) Multiple affinity domains for the detection, purification and immobilization of recombinant proteins. J. Mol. Recognit. 9(5-6), 585-594.

Nilsson, J., Ståhl S., Lundeberg J., Uhlén M., and Nygren, P. Å. (1997) Affinity fusion strategies for detection, purification and immobilization of recombinant proteins. Protein Expression Purif. 11, 1-16.

Nord, K., Gunneriusson, E., Ringdahl, J., Ståhl, S., Uhlén, M., and Nygren, P. Å. (1997) Binding proteins selected from combinatorial libraries of an alpha-helical bacterial receptor domain. Nat. Biotechnol. 15(8), 772-777.

Nygren, P. Å, and Uhlén, M. (1997) Scaffolds for engineering novel binding sites in proteins. Curr. Opin. Struct. Biol. 7(4), 463-469.

Rieker, J. D., and Hu, J. C. (2000) Molecular applications of fusions to leucine zippers. Methods Enzymol. 328, 282-296.

Sakurai, T., Roonprapunt, C., and Grumet, M. (1998) Purification of Ig-fusion proteins from medium containing Ig. Biotechniques 25(3), 382-385.

Saleemuddin M (1999) Bioaffinity based immobilization of enzymes. Adv. Biochem. Eng. Biotechnol. 64, 203-226.

Salminen, T. A., Smith, D. J., Jalkanen, S., and Johnson, M. S. (1998) Structural model of the catalytic domain of an enzyme with cell adhesion activity: human vascular adhesion protein-1 (HVAP-1) D4 domain is an amine oxidase. Protein Eng. 11(12), 1195-204.

Sheibani, N. (1999) Prokaryotic gene fusion expression systems and their use in structural and functional studies of proteins. Prep. Biochem. & Biotechnol. 29, 77-90.

Shpigel, E., Goldlust, A., Efroni, G., Avraham, A., Eshel, A., Dekel, M., and Shoseyov, O. (1999) Immobilization of recombinant heparinase I fused to cellulose-binding domain. Biotechnol. Bioeng. 65(1), 17-23.

Skerra, A. (2000) Engineered protein scaffolds for molecular recognition. J. Mol. Recognit. 13(4), 167-187.

Smith, D. B. and Johnson, K. S. (1988) Single-step purification of polypeptides expressed in Escerichia coli as fusions with glutathione S-transferase. Gene 67, 31-40.

Smith, D. J., Salmi, M., Bono, P., Hellman, J., Leu, T., and Jalkanen, S. (1998) Cloning of vascular adhesion protein 1 reveals a novel multifunctional adhesion molecule. J Exp. Med. 188, 17-27.

Tischer, W., and Kasche, V. (1999) Immobilized enzymes: crystals or carriers? Trends Biotechnol. 17(8), 326-335.

Tomme, P., Boraston, A., McLean, B., Kormos, J., Creagh, A. L., Sturch, K., Gilkes, N. R., Haynes, C. A., Warren, R. A., and Kilburn, D. G. (1998) Characterization and affinity applications of cellulose-binding domains. J. Chromatogr. B Biomed. Sci. Appl. 715(1), 283-296.

Tudyka, T., and Skerra, A. (1997) Glutathione S-transferase can be used as a C-terminal enzymatically active dimerization module for a recombinant protease inhibitor, and functionally secreted into the periplasm of Escherichia coli. Protein Sci. 6, 2180-2187.

Turkova, J. (1999) Oriented immobilization of biologically active proteins as a tool for revealing protein interactions and function. J. Chromatogr. B Biomed. Sci. Appl. 722(1-2), 11-31.

Uhlén, M., Forsberg, G., Moks T., Hartmanis, M., and Nilsson, B. (1992) Fusion proteins in Biotechnology. Curr. Opin. Biotechnol. 3, 363-369.

Wang, Q. M., Johnson, R. B., Cox, G. A., Villarreal, E. C., and Loncharich, R. J. (1997) A continuous colorimetric assay for rhinovirus-14 3C protease using peptide p-nitroanilides as substrates. Anal. Biochem. 252(2), 238-245.

Walker, P. A., Leong, L. E., Ng, P. W., Tan, S. H., Waller, S., Murphy, D., and Porter, A. G. (1994) Efficient and rapid affinity purification of proteins using recombinant fusion proteases. Bio/Technology 12, 601-605.

Wimmerova, M., and Macholan, L. (1999) Sensitive amperometric biosensor for the determination of biogenic and synthetic amines using pea seedlings amine oxidase: a novel approach for enzyme immobilisation. Biosens. Bioelectron. 14(8-9), 695-702.

Yu, P. H. and Zuo, D. M. (1993) Oxidative deamination of methylamine by semicarbazide-sensitive amine oxidase leads to cytotoxic damage in endothelial cells. Possible consequences for diabetes. Diabetes 42(4), 594-603.

Yu, P. H., Zuo, D. M., and Davis, B. A. (1994) Characterization of human serum and umbilical artery semicarbazide-sensitive amine oxidase (SSAO). Species heterogeneity and stereoisomeric specificity. Biochem. Pharmacol. 47(6), 1055-1059.

Zhang, X., Kim, J., and McIntire, W. S. (1995) cDNA sequences of variant forms of human placenta diamine oxidase. Biochem. Genet. 33(7-8), 261-268.

Zhang, X and McIntire, W. S. (1996) Cloning and sequencing of a copper-containing, topa quinone-containing monoamine oxidase from human placenta. Gene 179, 279-286.

What is claimed is:

1. A nucleic acid comprising a nucleotide sequence encoding a secreted fusion protein comprising:
(i) a signal peptide that directs secretion of the fusion protein from a host cell;
(ii) a soluble form of human semicarbazide-sensitive amine oxidase (SSAO);
(iii) a fusion partner that enables dimerization of the soluble form of human SSAO; and
(iv) a protease cleavage site located between the soluble form of human SSAO and the fusion partner.

2. The nucleic acid according to claim 1, wherein the soluble form of human SSAO comprises amino acids 29 to 763 of SEQ ID NO: 2 or a fragment thereof.

3. The nucleic acid according to claim 2, wherein the fusion protein has benzylamine oxidase activity.

4. The nucleic acid according to claim 2, wherein the soluble form of human SSAO comprises amino acids 29 to 763 of SEQ ID NO: 2.

5. The nucleic acid according to claim 1, wherein the fusion protein lacks the membrane spanning portion of human SSAO.

6. The nucleic acid according to claim 1, wherein the fusion protein lacks amino acids 6 to 26 of SEQ ID NO: 2.

7. The nucleic acid according to claim 1, wherein the fusion partner is fused to the N-terminal portion of the soluble form of human SSAO.

8. The nucleic acid according to claim 1, wherein the fusion partner is glutathione S-transferase or a functionally equivalent variant thereof.

9. The nucleic acid according to claim 8, wherein the fusion partner is a variant of Schistosoma japonicum glutathione S-transferase, the variant having at least one of the cysteine residues in positions 85, 138, and 178 replaced by another amino acid residue.

10. The nucleic acid according to claim 8, wherein the fusion partner comprises the amino acid sequence of SEQ ID NO: 4 or SEQ ID NO: 5.

11. The nucleic acid according to claim 1, wherein the signal peptide is a mouse IgG1 heavy chain signal peptide.

12. The nucleic acid according to claim 1, wherein the protease cleavage site is a 3C protease cleavage site.

13. nucleic acid according to claim 12, wherein the 3 C protease cleavage site comprises the amino acid sequence EALFQG (SEQ ID NO: 6).

14. The nucleic acid according to claim 1, wherein the fusion protein comprises the amino acid sequence of SEQ ID NO: 20.

15. An expression vector comprising the nucleic acid of claim 1.

16. An expression vector comprising the nucleic acid of claim 14.

17. A method for the purification of a recombinant human SSAO, the method comprising:
(i) transfecting a cell with the expression vector according to claim 15;
(ii) culturing the cell in a culture medium and under conditions wherein the fusion protein encoded by the expression vector is secreted into the culture medium;
(iii) binding the secreted fusion protein to a ligand having affinity for the fusion partner;
(iv) separating the fusion partner and the soluble form of human SSAO; and
(v) recovering the soluble form of human SSAO.

18. The method according to claim 17, wherein the ligand having affinity for the fusion partner is glutathione or a derivative thereof.

19. The method according to claim 17, wherein the fusion partner is separated from the soluble form of human SSAO by protease cleavage.

20. The method according to claim 19, wherein the protease is a picornavirus 3C-protease.

21. The method according to claim 20, wherein the protease is rhinovirus 3C-protease.

22. The method according to claim 19, wherein the protease is fused to a fusion partner resulting in a fusion protease.

23. The method according to claim 22, wherein the fusion protease is separated from the soluble form of human SSAO by a process comprising binding the fusion protease to a ligand having affinity for the fusion protease.

24. A method for the preparation of an immobilized recombinant human SSAO, the method comprising:
(i) transfecting a cell with the expression vector according to claim 15;
(ii) culturing the cell in a culture medium and under conditions wherein the fusion protein encoded by the expression vector is secreted into the culture medium; and
(iii) binding the secreted fusion protein to a ligand having affinity for the fusion partner to thereby immobilize the fusion protein.

25. A fusion protein encoded by the nucleic acid of claim 1.

26. The fusion protein of claim 25, wherein the fusion protein is immobilized on a ligand having affinity for the fusion partner.


SEQUENCE LISTING (20 sequences)

SEQ ID NO: 1 - DNA, 4040 bp, Homo sapiens
  CDS (161)...(2449)
gtccttcccacccttagtcccaggcatctgactaccgggaacctcagccagagtccgggagccccccaccccgtccaggagccaacagagcccccgtcttgctggcgtgagaatacattgctctcctttggttgaatcagctgtccctcttcgtgggaaaatgaaccagaagacaatcctcgtgctcctcattctggccgtcatcaccatctttgccttggtttgtgtcctgctggtgggcaggggtggagatgggggtgaacccagccagcttccccattgcccctctgtatctcccagtgcccagccttggacacaccctggccagagccagctgtttgcagacctgagccgagaggagctgacggctgtgatgcgctttctgacccagcggctggggccagggctggtggatgcagcccaggcccggccctcggacaactgtgtcttctcagtggagttgcagctgcctcccaaggctgcagccctggctcacttggacagggggagccccccacctgcccgggaggcactggccatcgtcttctttggcaggcaaccccagcccaacgtgagtgagctggtggtggggccactgcctcacccctcctacatgcgggacgtgactgtggagcgtcatggaggccccctgccctatcaccgacgccccgtgctgttccaagagtacctggacatagaccagatgatcttcaacagagagctgccccaggcttctgggcttctccaccactgttgcttctacaagcaccggggacggaacctggtgacaatgaccacggctccccgtggtctgcaatcaggggaccgggccacctggtttggcctctactacaacatctcgggcgctgggttcttcctgcaccacgtgggcttggagctgctagtgaaccacaaggcccttgaccctgcccgctggactatccagaaggtgttctatcaaggccgctactacgacagcctggcccagctggaggcccagtttgaggccggcctggtgaatgtggtgctgatcccagacaatggcacaggtgggtcctggtccctgaagtcccctgtgcccccgggtccagctccccctctacagttctatccccaaggcccccgcttcagtgtccagggaagtcgagtggcctcctcactgtggactttctcctttggcctcggagcattcagtggcccaaggatctttgacgttcgcttccaaggagaaagactagtttatgagataagcctccaagaggccttggccatctatggtggaaattccccagcagcaatgacgacccgctatgtggatggaggctttggcatgggcaagtacaccacgcccctgacccgtggggtggactgcccctacttggccacctacgtggactggcacttccttttggagtcccaggcccccaagacaatacgtgatgccttttgtgtgtttgaacagaaccagggcctccccctgcggcgacaccactcagatctctactcgcactactttgggggtcttgcggaaacggtgctggtcgtcagatctatgtccaccttgctcaactatgactatgtgtgggatacggtcttccaccccagtggggccatagaaatacgattctatgccacgggctacatcagctcggcattcctctttggtgctactgggaagtacgggaaccaagtgtcagagcacaccctgggcacggtccacacccacagcgcccacttcaaggtggatctggatgtagcaggactggagaactgggtctgggccgaggatatggtctttgtccccatggctgtgccctggagccctgagcaccagctgcagaggctgcaggtgacccggaagctgctggagatggaggagcaggccgccttcctcgtgggaagcgccacccctcgctacctgtacctggccagcaaccacagcaacaagtggggtcacccccggggctaccgcatccagatgctcagctttgctggagagccgctgccccaaaacagctccatggcgagaggcttcagctgggagaggtaccagctggctgtgacccagcggaaggaggaggagcccagtagcagcagcgttttcaatcagaatgacccttgggcccccactgtggatttcagtgacttcatcaacaatgagaccattgctggaaaggatttggtggcctgggtgacagctggttttctgcatatcccacatgcagaggacattcctaacacagtgactgtggggaacggcgtgggcttcttcctccgaccctataacttctttgacgaagacccctccttctactctgccgactccatctacttccgaggggaccaggatgctggggcctgcgaggtcaaccccctagcttgcctgccccaggctgctgcctgtgcccccgacctccctgccttctcccacgggggcttctctcacaactaggcggtcctgggatggggcatgtggccaagggctccagggccagggtgtgagggatggggagcagctgggcactgggccggcagcctggttccctctttcctgtgccaggactctctttcttccactaccctccctcgcatccgcctctgagccaggagcctcctgaccctgtgatgcctgacacaggggacactgaaccttgttgatgccagctgtactgagttctcatccacagaggccaggcatggcccagcctggagccgtggccgagggcttccctagatggttccctttgttgctgtctggctttcccgaatctttttaggccacctccaaggactctaaaagggggctattccctggagaccccagagtagggttgccagtcctgcaagtccatagctgagctggaaaggatgcttctgctcacattccctctcatccaggtcctttccttctcgtcttcctctctctcacctacttcctcctcctcctcctgttcctgccttctcttctatcctgcaatttctcccgaatcctgaggggatatccctatgtcccagcccctggtactcccccagccctcagttttcagtcaagttccgtctcctctccagccctatggaagtctcaaggtcacgggacccctaatcagagtggccaatccctgtgtgtcgttcccttgtgtctgttgcttattgggagtaggagttgctcctacccctgtcctggggctgggtgtgtttcaggacagctgcttctgtgcatttgtgtctgcctgcctcatgctctctatagaggaggatggtcatcgtgacagcagcagctcaagttagcatttcaagtgatttgggggtgcaatgataatgaagaatggccattttgtaccagggctctgtattctgcaacagcctgtttgggaggctggagtggaaacaaagggtgggcatcaaagatgagaagccaaagcccctacaactccagccacccagccaggaggggctgtccaatcacattcaggcatgcgaatgagctgggccctgggtgaggtgggggtctggcctagtggggaggggcctggcctgggtggggcagggcctggcctggtccaggcttgggctccattcccatcactgctgtccctcctgaggtctggattggggatggggacaaagaaatagcaagagatgagaaacaacagaaacttttttctctaaaggactggttaaatcaattctgatacagccttacaatacaatagtatgcagctaaaaaataattgtatgtctttatatactaatatgtaataatcttcaggtgaaaaaggcaagccacagaaatgtgtatagcgcacttcccatttgtgtttcagaaaggagtagaatataaacacataattgcttatgtatgcctattcagaataaatgggtaacactgattacttttgggaggggaaccagtaggttgaggacaggagagggaagggtcttaacacttacacccttttgtacattttgaattttgaaccatgtgactgtattacctattcaaaataaacaataaatgggcccaaaaaaaaaaaaaaaaa

SEQ ID NO: 2 - protein, 763 aa, Homo sapiens
MNQKTILVLLILAVITIFALVCVLLVGRGGDGGEPSQLPHCPSVSPSAQPWTHPGQSQLFADLSREELTAVMRFLTQRLGPGLVDAAQARPSDNCVFSVELQLPPKAAALAHLDRGSPPPAREALAIVFFGRQPQPNVSELVVGPLPHPSYMRDVTVERHGGPLPYHRRPVLFQEYLDIDQMIFNRELPQASGLLHHCCFYKHRGRNLVTMTTAPRGLQSGDRATWFGLYYNISGAGFFLHHVGLELLVNHKALDPARWTIQKVFYQGRYYDSLAQLEAQFEAGLVNVVLIPDNGTGGSWSLKSPVPPGPAPPLQFYPQGPRFSVQGSRVASSLWTFSFGLGAFSGPRIFDVRFQGERLVYEISLQEALAIYGGNSPAAMTTRYVDGGFGMGKYTTPLTRGVDCPYLATYVDWHFLLESQAPKTIRDAFCVFEQNQGLPLRRHHSDLYSHYFGGLAETVLVVRSMSTLLNYDYVWDTVFHPSGAIEIRFYATGYISSAFLFGATGKYGNQVSEHTLGTVHTHSAHFKVDLDVAGLENWVWAEDMVFVPMAVPWSPEHQLQRLQVTRKLLEMEEQAAFLVGSATPRYLYLASNHSNKWGHPRGYRIQMLSFAGEPLPQNSSMARGFSWERYQLAVTQRKEEEPSSSSVFNQNDPWAPTVDFSDFINNETIAGKDLVAWVTAGFLHIPHAEDIPNTVTVGNGVGFFLRPYNFFDEDPSFYSADSIYFRGDQDAGACEVNPLACLPQAAACAPDLPAFSHGGFSHN

SEQ ID NO: 3 - DNA, 739 bp, Schistosoma japonicum
  CDS (17)...(670)
tttaggtaacttggtcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaataaattaagaatgattgttttagtaaacattatttatcacttacaattaaactaaatataaatgtcgac

SEQ ID NO: 4 - protein, 218 aa, Schistosoma japonicum
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 5 - protein, 218 aa, Schistosoma japonicum
MSPILGYWKIKGLVQPTRLLLEYLEEKYEEHLYERDEGDKWRNKKFELGLEFPNLPYYIDGDVKLTQSMAIIRYIADKHNMLGGSPKERAEISMLEGAVLDIRYGVSRIAYSKDFETLKVDFLSKLPEMLKMFEDRLSHKTYLNGDHVTHPDFMLYDALDVVLYMDPMCLDAFPKLVSFKKRIEAIPQIDKYLKSSKYIAWPLQGWQATFGGGDHPPK

SEQ ID NO: 6 - protein, 6 aa, Artificial Sequence
  Protease cleavage site
EALFQG

SEQ ID NO: 7 - DNA, 43 bp, Artificial Sequence
  PCR primer
ccggaattccaacgcgtccatgaaccagaagacaatcctcgtg

SEQ ID NO: 8 - DNA, 45 bp, Artificial Sequence
  PCR primer
cccccaagcttgtcgactcactagttgtgagagagaagccccccc

SEQ ID NO: 9 - DNA, 36 bp, Artificial Sequence
  PCR primer
gaggaagctttgttccaaggtggagatgggggtgaa

SEQ ID NO: 10 - DNA, 21 bp, Artificial Sequence
  PCR primer
gcattctagttgtggtttgtc

SEQ ID NO: 11 - DNA, 37 bp, Artificial Sequence
  PCR primer
gccggaattcgacgcgtcccctatactaggttattgg

SEQ ID NO: 12 - DNA, 37 bp, Artificial Sequence
  PCR primer
ctctgcgcgctcttttggagaacccaacatgttgtgc

SEQ ID NO: 13 - DNA, 40 bp, Artificial Sequence
  PCR primer
ggttctccaaaagagcgcgcagagatttcaatgcttgaag

SEQ ID NO: 14 - DNA, 36 bp, Artificial Sequence
  PCR primer
atgagataaacggtcttcgaacattttcagcatttc

SEQ ID NO: 15 - DNA, 44 bp, Artificial Sequence
  PCR primer
gttcgaagaccgtttatctcataaaacatatttaaatggtgatc

SEQ ID NO: 16 - DNA, 33 bp, Artificial Sequence
  PCR primer
aaaagaaactagttttgggaacgcatccaggca

SEQ ID NO: 17 - DNA, 40 bp, Artificial Sequence
  PCR primer
cccaaaactagtttcttttaaaaaacgtattgaagctatc

SEQ ID NO: 18 - DNA, 44 bp, Artificial Sequence
  PCR primer
acccaagcttcctgactttgtgactttggaggatggtcgccacc

SEQ ID NO: 19 - DNA, 3006 bp, Artificial Sequence
  Recombinant construct
atggattggctgcggaacttgctattcctgatggcggccgctcaaagtatcaacgccgcgcaacacgatgaagccgtagacaacaaattcaacaaagaacaacaaaacgcgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttctccaaaagagcgcgcagagatttcaatgcttgaaggagcggttttggatattagatacggtgtttcgagaattgcatatagtaaagactttgaaactctcaaagttgattttcttagcaagctacctgaaatgctgaaaatgttcgaagaccgtttatctcataaaacatatttaaatggtgatcatgtaacccatcctgacttcatgttgtatgacgctcttgatgttgttttatacatggacccaatgtgcctggatgcgttcccaaaactagtttcttttaaaaaacgtattgaagctatcccacaaattgataagtacttgaaatccagcaagtatatagcatggcctttgcagggctggcaagccacgtttggtggtggcgaccatcctccaaagtcacaaagtcaggaagctttgttccaaggtggagatgggggtgaacccagccagcttccccattgcccctctgtatctcccagtgcccagccttggacacaccctggccagagccagctgtttgcagacctgagccgagaggagctgacggctgtgatgcgctttctgacccagcggctggggccagggctggtggatgcagcccaggcccggccctcggacaactgtgtcttctcagtggagttgcagctgcctcccaaggctgcagccctggctcacttggacagggggagccccccacctgcccgggaggcactggccatcgtcttctttggcaggcaaccccagcccaacgtgagtgagctggtggtggggccactgcctcacccctcctacatgcgggacgtgactgtggagcgtcatggaggccccctgccctatcaccgacgccccgtgctgttccaagagtacctggacatagaccagatgatcttcaacagagagctgccccaggcttctgggcttctccaccactgttgcttctacaagcaccggggacggaacctggtgacaatgaccacggctccccgtggtctgcaatcaggggaccgggccacctggtttggcctctactacaacatctcgggcgctgggttcttcctgcaccacgtgggcttggagctgctagtgaaccacaaggcccttgaccctgcccgctggactatccagaaggtgttctatcaaggccgctactacgacagcctggcccagctggaggcccagtttgaggccggcctggtgaatgtggtgctgatcccagacaatggcacaggtgggtcctggtccctgaagtcccctgtgcccccgggtccagctccccctctacagttctatccccaaggcccccgcttcagtgtccagggaagtcgagtggcctcctcactgtggactttctcctttggcctcggagcattcagtggcccaaggatctttgacgttcgcttccaaggagaaagactagtttatgagataagcctccaagaggccttggccatctatggtggaaattccccagcagcaatgacgacccgctatgtggatggaggctttggcatgggcaagtacaccacgcccctgacccgtggggtggactgcccctacttggccacctacgtggactggcacttccttttggagtcccaggcccccaagacaatacgtgatgccttttgtgtgtttgaacagaaccagggcctccccctgcggcgacaccactcagatctctactcgcactactttgggggtcttgcggaaacggtgctggtcgtcagatctatgtccaccttgctcaactatgactatgtgtgggatacggtcttccaccccagtggggccatagaaatacgattctatgccacgggctacatcagctcggcattcctctttggtgctactgggaagtacgggaaccaagtgtcagagcacaccctgggcacggtccacacccacagcgcccacttcaaggtggatctggatgtagcaggactggagaactgggtctgggccgaggatatggtctttgtccccatggctgtgccctggagccctgagcaccagctgcagaggctgcaggtgacccggaagctgctggagatggaggagcaggccgccttcctcgtgggaagcgccacccctcgctacctgtacctggccagcaaccacagcaacaagtggggtcacccccggggctaccgcatccagatgctcagctttgctggagagccgctgccccaaaacagctccatggcgagaggcttcagctgggagaggtaccagctggctgtgacccagcggaaggaggaggagcccagtagcagcagcgttttcaatcagaatgacccttgggcccccactgtggatttcagtgacttcatcaacaatgagaccattgctggaaaggatttggtggcctgggtgacagctggttttctgcatatcccacatgcagaggacattcctaacacagtgactgtggggaacggcgtgggcttcttcctccgaccctataacttctttgacgaagacccctccttctactctgccgactccatctacttccgaggggaccaggatgctggggcctgcgaggtcaaccccctagcttgcctgccccaggctgctgcctgtgcccccgacctccctgccttctcccacgggggcttctctcacaactagtgagtcgac

SEQ ID NO: 20 - protein, 998 aa, Artificial Sequence [sequence omitted]